SEQUENCE LISTING (18 sequences)

SEQ ID NO: 1 - protein, 404 aa, Artificial sequences
  Gryllacrididae
EGSGAASGAKTVSVAGTDGSGGALAGSAAGAISKASGDEGSGAASGAKTVSVAGTDGSEGAVAGGVAGASSKASGNEGSGAASGAKTVSVAGTDGSGGALAGGAAGASSEASGDEGSGAASGAKTVSVAETDGSGGALAGSAAGASSKASGDEGSGAASGAKTVSVAGTDGSEGAVAGSAAGASSKASGNEGSGAASGAKTVSVAGTDGSGGAVAGGTAGASSIASGDGGSGAASRANTVSVAGSDGSGGAIAGSSADSSGIAVGDGAALSGSSALARSASDGAGAAVGAAQAGSVATGSGSDDSFAATNAEAGTISYGDGSLSAAQSGSIAPAKGADVNVQSGSISSAGNSVGAAGGSVETGQSHHCKKPVSTNSQPGGSGAEVNLPGLRIKSDAVATVGLVL

SEQ ID NO: 2 - protein, 427 aa, Artificial sequence
  Gryllacrididae
VAGTDGSGGALARGAAGASSVASGDGAGAASGAKTVSVAGTDGSEGAVAGGVAGASSKASGNEGSGAASGAKTVSVAGTDGSGGALAGSAAGAISKASGDEGSGAASGAKTVSVAGTDGSEGAVAVGVAGASSKASGNEGSGAASGAKTVSVAGTDGSGGAHAGSAAGASSKASGDEGSGAASGAKTVSVAGTDGSGGALAGGAAGASSEASGDEGSGAASGAKAVSVAGTDGSGGALARGAAGASSVASGDGAGAASRANAGSIAGSGGPRGAVSGSSADVSSIAVGDGAALSGSSALARSASDGAGAAVGAAQAGSVATGSGSDDSFAATNAEAGTISYGDGSLSAAQSGSIAAAKGADVNVQSGSISSAGNSVGAAGGSVETGQSHHCKRPVSTNSQPGGSGAEVNLPGLRIKSDAVATVGLVL

SEQ ID NO: 3 - protein, 425 aa, Artificial sequence
  Gryllacrididae
MDFKIFLLVLLGAVVVLSAVEGRPSGDDDVRIISSTKDVTSTGKRRSNSKNNVAQSSSQKDSIRVKDSNEVIKSISENDPDGTRITTSKGTGVGSETLNENLVELNQNTVSDTNSVETSSDKSNSVTTYDADDRPLTVTDRKGQKDVKSATNAKEDSSEVSENSESETNWQSESDSVEKSVSHPVCGGAESPSSGGPQGSGPQGGGSQGGPSGQGQPNGGIAVSSAEDAAAPQSTRTSTAEGESSSFSAAADVNEGSRKGNVALAGSTAYGRSVTNENSSSGSTQSFSGAAEENVASPDGGSTQRNAAIAGNIAEGQVSSKEEVAEGKATSLSGTIQESDENSQRSAAIAGSSAEANVATREQTSKGKTAAWTGNSQEKSTSPAEISETNIFSSGSYAEGSTLSREKLEQGKSTTWSEESEESRK

SEQ ID NO: 4 - protein, 226 aa, Artificial sequence
  Gryllacrididae
MGSSALYLATIFLSLQVIDRSSAVAISLPRRIYPRSRPAFSLAETKILDEICSQSQCKDSCYGCFSKLSGEQQSSESLKELKKCSSTYLGKSPYSKCQKILDNGSFNGKCDTNSTVFCDYENCLLNIELDSLVAECKLEAKADGANIFKGTTICILTRIRCREVRFSTGYYYSPNFNYALSFNKDLKLQLFPASYLPSDGRLTCGSQDPYQPPSQAAWRNRTCDLC

SEQ ID NO: 5 - protein, 529 aa, Artificial sequence
  Gryllacrididae
KWKWLPKKRRLRSRKLHPQLGIPSNVFSGSPLDVLSQGPVAPGESLNAVPGSPTGFNQGSGLFPHSPLDGSSAQGSPSAGQSPQESPISSSNLIPLLGSSPDDLGEEYYEVEEDDGSWDYVSPDENTNSEDEHEDSSEDTNSSLPGSSGSPPEGPSSGPNSPSGNTETGSPKNENGNSPQSRSQSSVPSLAPSSPSGSTSSPIVGKPEGDTSSSGGPSGSASPQVSSPGQSSQGTPGSLSGTPQDGSLSPGSPQDASSPEQSSQGAPGSSTDAPQDSSGSATKDDSPSSNGRTSKPSSATKRPGKGIKGLTKKKKGSPKAGKWTWLRKPGKRLRKKLKPVQLKPSGIPLSQGILKPTTDVSPNVIAQGNAAASAVAEGGKLSPNAPSPRSIAASNADANAAISSPDASLRADSAASSVASQVNNRSPLGALQGSRAEADAAAQAVITAPNQPRQEPRVSPGSAQEKSQRGNPFDLRNSLWQDENDQFNSLSSKLNQLSKNNKHLASNINNIENIFDKLYKFRSNTATNK

SEQ ID NO: 6 - protein, 190 aa, Artificial Sequence
  polypeptide
EGSGAASGAKTVSVAGTDGSGGALAGSAAGAISKASGDEGSGAASGAKTVSVAGTDGSEGAVAGGVAGASSKASGNEGSGAASGAKTVSVAGTDGSGGALAGGAAGASSEASGDEGSGAASGAKTVSVAETDGSGGALAGSAAGASSKASGDEGSGAASGAKTVSVAGTDGSEGAVAGSAAGASSKASGN

SEQ ID NO: 7 - protein, 214 aa, Artificial Sequence
  polypeptide
EGSGAASGAKTVSVAGTDGSGGAVAGGTAGASSIASGDGGSGAASRANTVSVAGSDGSGGAIAGSSADSSGIAVGDGAALSGSSALARSASDGAGAAVGAAQAGSVATGSGSDDSFAATNAEAGTISYGDGSLSAAQSGSIAPAKGADVNVQSGSISSAGNSVGAAGGSVETGQSHHCKKPVSTNSQPGGSGAEVNLPGLRIKSDAVATVGLVL

SEQ ID NO: 8 - protein, 189 aa, Artificial Sequence
  polypeptide
VAGTDGSGGALARGAAGASSVASGDGAGAASGAKTVSVAGTDGSEGAVAGGVAGASSKASGNEGSGAASGAKTVSVAGTDGSGGALAGSAAGAISKASGDEGSGAASGAKTVSVAGTDGSEGAVAVGVAGASSKASGNEGSGAASGAKTVSVAGTDGSGGAHAGSAAGASSKASGDEGSGAASGAKTVS

SEQ ID NO: 9 - protein, 238 aa, Artificial Sequence
  polypeptide
VAGTDGSGGALAGGAAGASSEASGDEGSGAASGAKAVSVAGTDGSGGALARGAAGASSVASGDGAGAASRANAGSIAGSGGPRGAVSGSSADVSSIAVGDGAALSGSSALARSASDGAGAAVGAAQAGSVATGSGSDDSFAATNAEAGTISYGDGSLSAAQSGSIAAAKGADVNVQSGSISSAGNSVGAAGGSVETGQSHHCKRPVSTNSQPGGSGAEVNLPGLRIKSDAVATVGLVL

SEQ ID NO: 10 - protein, 261 aa, Artificial Sequence
  polypeptide
MDFKIFLLVLLGAVVVLSAVEGRPSGDDDVRIISSTKDVTSTGKRRSNSKNNVAQSSSQKDSIRVKDSNEVIKSISENDPDGTRITTSKGTGVGSETLNENLVELNQNTVSDTNSVETSSDKSNSVTTYDADDRPLTVTDRKGQKDVKSATNAKEDSSEVSENSESETNWQSESDSVEKSVSHPVCGGAESPSSGGPQGSGPQGGGSQGGPSGQGQPNGGIAVSSAEDAAAPQSTRTSTAEGESSSFSAAADVNEGSRKGN

SEQ ID NO: 11 - protein, 164 aa, Artificial Sequence
  polypeptide
VALAGSTAYGRSVTNENSSSGSTQSFSGAAEENVASPDGGSTQRNAAIAGNIAEGQVSSKEEVAEGKATSLSGTIQESDENSQRSAAIAGSSAEANVATREQTSKGKTAAWTGNSQEKSTSPAEISETNIFSSGSYAEGSTLSREKLEQGKSTTWSEESEESRK

SEQ ID NO: 12 - protein, 321 aa, Artificial Sequence
  polypeptide
KWKWLPKKRRLRSRKLHPQLGIPSNVFSGSPLDVLSQGPVAPGESLNAVPGSPTGFNQGSGLFPHSPLDGSSAQGSPSAGQSPQESPISSSNLIPLLGSSPDDLGEEYYEVEEDDGSWDYVSPDENTNSEDEHEDSSEDTNSSLPGSSGSPPEGPSSGPNSPSGNTETGSPKNENGNSPQSRSQSSVPSLAPSSPSGSTSSPIVGKPEGDTSSSGGPSGSASPQVSSPGQSSQGTPGSLSGTPQDGSLSPGSPQDASSPEQSSQGAPGSSTDAPQDSSGSATKDDSPSSNGRTSKPSSATKRPGKGIKGLTKKKKGSPKAG

SEQ ID NO: 13 - protein, 208 aa, Artificial Sequence
  polypeptide
KWTWLRKPGKRLRKKLKPVQLKPSGIPLSQGILKPTTDVSPNVIAQGNAAASAVAEGGKLSPNAPSPRSIAASNADANAAISSPDASLRADSAASSVASQVNNRSPLGALQGSRAEADAAAQAVITAPNQPRQEPRVSPGSAQEKSQRGNPFDLRNSLWQDENDQFNSLSSKLNQLSKNNKHLASNINNIENIFDKLYKFRSNTATNK

SEQ ID NO: 14 - DNA, 68 bp, Bacillus subtilis
aatttttaggataatatacaaaatcccccttacttcgacaattgcaatctggtattatcgtatcgcat

SEQ ID NO: 15 - DNA, 50 bp, Bacillus subtilis
ataccatttacatccaattaacatccgtctgctaaactgactggcatagg

SEQ ID NO: 16 - DNA, 45 bp, Bacillus subtilis
attcacacttcttaacataccatttacatccaattaacatccgtc

SEQ ID NO: 17 - DNA, 50 bp, Bacillus subtilis
ttcggttcaaaccctttttacatagaacctttactctatacgtgtaggac

SEQ ID NO: 18 - DNA, 51 bp, Bacillus subtilis
cactgatttacaaaaccttaacattcggttcaaaccctttttacatagaac

(19) United States
(12) Patent Application Publication
(10) Pub. No.: US 2020/0208343 A1
(43) Pub. Date: Jul. 2, 2020

(54) SELF-HEALING FABRIC

(71) Applicant: AUGMANITY NANO LTD., Rehovot (IL)

(72) Inventors: Neta RAAB, Hod Hasharon (IL); Ido BACHELET, Tel-Aviv (IL)

(21) Appl. No.: 16/633,043

(22) PCT Filed: Jul. 22, 2018
(86) PCT No.: PCT/IL2018/050812

§ 371 Date: Jan. 22, 2020

Related U.S. Application Data
(60) Provisional application No. 62/535,989, filed on Jul. 24, 2017.
(60) Provisional application No. 62/540,069, filed on Aug. 2, 2017.

(51) Int. Cl. D06M 16/00; C12N 15/03; C07K 14/32; C12N 11/02

(52) U.S. Cl. CPC D06M 16/003; C12N 11/02; C07K 14/32; C12N 15/03

(57) ABSTRACT

The present invention provides polynucleotide constructs with at least one disruption or tearing responsive promoter and at least one sequence encoding a fiber-forming protein. The invention further provides a cell comprising such a construct as well as a biofilm containing a plurality of such cells. A biofilm comprising cells containing the construct of the present invention are capable of forming protein fibers in response to a disruption stimulus that are useful, for example, in preparation of self-healing fabrics and textiles.

### FIELD OF THE INVENTION

The present invention relates to polynucleotide constructs comprising at least one promoter responsive to tearing or disruption of a biofilm and at least one sequence encoding a fiber-forming protein, to a biofilm comprising cells containing said construct and use of said biofilm in production of a self-healing fabric.

### BACKGROUND OF THE INVENTION

In contrast to living organisms, most everyday objects lack the ability to respond to damage and self-heal or regenerate. In cases where the cost of fixing an object is higher than the cost of replacing it, the latter option is usually preferred. This arrangement may have very negative long-term environmental impact.

Self-healing or self-recovery are highly complex properties based on two subsequent phases: a sensory phase, in which a certain threshold of breach of structural integrity is detected by the system; and a synthetic phase, in which synthesis of new material is driven by breach detection. For some products, such as textile articles, sensing per se may be easily achieved (e.g. by integrating conducting fibers into textile such that tearing would result in a measureable change in resistivity of a fabric segment). Synthesis and its coupling to sensing is more challenging, especially if the purpose is achieving the continuous, low maintenance ability to self-recover, which does not require constant refilling and tuning of fabric monomer reservoirs.

Although self-healing and regeneration are critical components of sustainable textiles, reports on achieving them in artificial textile systems is extremely scarce. Gaddes reported a polyelectrolyte layer-by-layer film coupled to squid ring proteins as a textile capable of suturing tears (Gaddes et al., ACS Applied Materials & Interfaces 8(31), 2016: 20371-20378.). Several reports demonstrated fabrics capable of restoring their protective hydrophobic coating (Xue et al., Scientific reports, 6, 2016: 27262).

There is an unmet need for development of methods allowing prolongation of the life expectancy of textile and clothing and in particular such methods allowing their self-healing.

### SUMMARY OF THE INVENTION

It is disclosed herein that a biofilm, engineered to express silk fibers upon disruption or tearing, when adsorbed or interlaced with a fabric is capable of repairing the fabric upon its physical tearing or disruption. Upon tearing, the biofilm expresses fiber-forming protein(s) that form protein fibers that seal the tear thereby healing the rupture. The present invention provides DNA constructs comprising at least one promoter responsive to mechanical tearing or disruption of a biofilm and at least one sequence encoding a fiber-forming protein. The invention further provides cells comprising said DNA construct, such as bacterial cells, capable of forming a biofilm. The invention further provides a biofilm comprising said cells comprising said construct and use of said biofilm in production of a self-healing fabric.

According to one aspect the present invention provides a DNA construct comprising a nucleic acid sequence of at least one promoter operably linked to at least one protein encoding sequence, wherein said at least one promoter has a nucleic acid sequence selected from the group consisting of SEQ ID NO: 17, 14, 15, 16, 18 and homologs thereof, and said at least one protein encoding sequence encodes a protein having an amino acid sequence selected from the group consisting of SEQ ID NOs: 1, 2, 3, 4, 5, 6, 7, 8, 9, 10, 11, 12, 13 and analogs thereof. According to some embodiments, the homolog and the analogue have at least 90% sequence identity to the parent sequence. According to one embodiment, the homolog and the analogue have at least 95% sequence identity to the parent sequence.

According to some embodiments, the protein encoding sequence encodes a protein having the amino acid sequence selected from the group consisting of SEQ ID NOs: 1, 2, 3, 4, 5, and analog thereof.

According to some specific embodiments, the DNA construct comprises a promoter having SEQ ID NO: 17 or a homolog thereof operably linked to a protein encoding sequence encoding a protein having the sequence set forth in SEQ ID NO. 1 or an analog thereof. According to one embodiment, the DNA construct comprises a promoter having SEQ ID NO: 17 operably linked to a protein encoding sequence encoding a protein having an amino acid sequence set forth in SEQ ID NO: 1.

According to specific embodiments, the DNA construct encodes a protein or proteins capable of self-assembling or assembling with each other to form at least one fiber. According to some embodiments, the protein that forms fibers upon self-assembly or upon assembly with each other has a sequence selected from SEQ ID NOs: 1-13.

According to another aspect, the present invention provides a vector comprising the DNA construct comprising a sequence of at least one promoter operably linked to at least one protein encoding sequence, wherein said at least one promoter has a nucleic acid sequence selected from the group consisting of SEQ ID NO: 17, 14, 15, 16, 18 and homologs thereof, and said at least one protein encoding sequence encodes a protein having the amino acid sequence selected from the group consisting of SEQ ID NOs: 1, 2, 3, 4, 5, 6, 7, 8, 9, 10, 11, 12, 13 and analog thereof.

According to various embodiments, the vector is a plasmid or phage.

According to a further aspect, the present invention provides a cell comprising the DNA construct comprising a nucleic acid sequence of at least one promoter operably linked to at least one protein encoding sequence, wherein said at least one promoter has a nucleic acid sequence selected from the group consisting of SEQ ID NO: 17, 14, 15, 16, 18 and homologs thereof, and said at least one protein encoding sequence encodes a protein having the amino acid sequence selected from the group consisting of SEQ ID NOs: 1, 2, 3, 4, 5, 6, 7, 8, 9, 10, 11, 12, 13 and analog thereof.

According to another aspect, the present invention provides a cell comprising a vector comprising said DNA construct.

According to certain embodiments, the cell comprising the DNA construct according to the invention or the vector comprising the DNA construct of the invention is a bacterial cell.

According to one aspect, the bacterial cell is capable of forming a biofilm containing a plurality of said bacterial cells.

According to another aspect, the present invention provides a biofilm comprising a plurality of cells according to the present invention. According to some embodiments, the biofilm expresses at least one of said encoded proteins upon tearing or disruption of said biofilm. According to some embodiments, the biofilm forms one or more protein fiber upon disruption of said biofilm.

According to some aspects, the present invention provides a fabric, wherein said fabric incorporates the cells or the biofilm of the present invention. According to some embodiments, the fabric comprises the biofilm according to the present invention wherein the biofilm is adsorbed to the surface of the fabric, or interlaced with the fabric. According to some embodiments, the fabric is a textile. According to some embodiments, the fabric is a textile made of natural fibers, synthetic fibers or blends thereof. According to some embodiments, the fabric is a textile selected from the group consisting of cotton, silk, wool, cashmere, linen, hemp, ramie, and jute.

According to an additional aspect, the present invention provides a self-healing textile comprising a biofilm according to the principles of the invention.

### BRIEF DESCRIPTION OF DRAWINGS

FIG. 1 shows B. subtilis biofilm transcriptome response to mechanical tear and subsequent mounting of a reporter response. FIG. 1A—shows the most up- or down-regulated genes, in Log(2) of the fold change compared to untreated biofilm. Mean p-value for all changes was 0.007±0.014. FIG. 1B shows response of B. subtilis biofilms to tear, measured in relative luminescence units (RLU) derived from expression of the luxABCDE operon under the control of SEQ ID NO: 17 promoter. The maximal signal was achieved after 30 min. FIG. 1C shows kinetic measurement of the response to tear driven by SEQ ID NO: 17 (circles—SEQ ID NO: 17 reporter strain; square—wildtype strain (control)).

FIG. 2 shows plasmid map: pBS3Clux-pst-sigA.

FIG. 3 shows spsegI-V analysis and self-assembly. FIG. 3A shows calculated net charge on protein surface. FIG. 3B shows SEM images showing assembled protein fibers following concentration by dehydration and acidification to approximately the calculated isoelectric point. Size bars=100 μm.

FIG. 4 shows silk fiber structures of single segments and a combination product. FIGS. 4A-E show structure of fibers formed by proteins spsegI-V, respectively; FIG. 4F shows fibers formed by the combination of spsegII+spsegV (all scale bars, 1000 μm).

FIG. 5 shows plasmid map: pINsilkII comprising SEQ ID NO: 17 promoter and spsegII protein.

FIG. 6 shows regenerating fabric system. FIG. 6A shows representative SEM images of regenerating fabric at t=˜15 minutes following tear of fabric-biofilm hybrid. Arrowheads point at assembled silk fibers; stars mark metal net discs used for imaging and circles point at fabric fibers. FIG. 6B shows images of torn regenerating hybrids (top 3 panels) vs. torn hybrids made with wildtype biofilms (bottom 3 panels). All images were taken at tear region.

FIG. 6C shows Quantitation of imaging fields visualized by scanning electron microscopy containing silk fibers. Each of the groups scanned comprised of 3 biological samples (sham, wildtype biofilms; vector, engineered biofilms). All size bars=100 μm.

### DETAILED DESCRIPTION OF THE INVENTION

According to one aspect the present invention provides a nucleic acid construct comprising a nucleic acid sequence of at least one promoter operably linked to at least one protein encoding sequence, wherein said at least one promoter has a nucleic acid sequence selected from the group consisting of SEQ ID NO: 17, 14, 15, 16, 18 and homolog thereof, and wherein said at least one protein encoding sequence encodes a protein having the amino acid sequence selected from the group consisting of SEQ ID NOs: 1, 2, 3, 4, 5, 6, 7, 8, 9, 10, 11, 12, 13 and analogs thereof. According to one embodiment, the nucleic acid is DNA. Thus in one embodiment, the present invention provides a DNA construct comprising a nucleic acid sequence of at least one promoter operably linked to at least one protein encoding sequence, wherein said at least one promoter has a nucleic acid sequence selected from the group consisting of SEQ ID NO: 17, 14, 15, 16, 18 and homolog thereof, and wherein said at least one protein encoding sequence encodes a protein having the amino acid sequence selected from the group consisting of SEQ ID NOs: 1, 2, 3, 4, 5, 6, 7, 8, 9, 10, 11, 12, 13 and analogs thereof.

The term “DNA construct”, as used herein, refers to an artificially constructed segment of nucleic acid. It can be an isolated or integrated in another DNA molecule. Accordingly, a “recombinant DNA construct” is produced by laboratory methods.

The term “nucleic acid” refers to single stranded or double stranded sequence (polymer) of deoxyribonucleotides or ribonucleotides. In addition, the polynucleotide includes analogues of natural polynucleotides, unless specifically mentioned. According to an embodiment, the nucleic acid may be selected from the group consisting of deoxyribonucleic acid (DNA), ribonucleic acid (RNA), peptide nucleic acid (PNA), locked nucleic acid (LNA), and analogues thereof, but is not limited thereto. The term encompasses DNA, RNA, single stranded or double stranded and chemical modifications thereof. The term “polynucleotide” as used herein refers to a long nucleic acid comprising more than 150 nucleotides. The terms “nucleic acid” and “polynucleotide” are used interchangeably herein.

The term “promoter” as used herein refers to a regulatory sequence that initiates transcription of a downstream nucleic acid. The term “promoter” refers to a DNA sequence within a larger DNA sequence defining a site to which RNA polymerase may bind and initiate transcription. A promoter may include optional distal enhancer or repressor elements. According to some embodiments the promoter is heterologous promoter, i.e., occurring naturally to direct the expression of a nucleic acid derived from a gene other than the desired nucleic acid. As used according to the present invention the term “DNA construct comprising a sequence of a promoter X” and the term “DNA construct comprising X” are used interchangeably. Thus “DNA construct comprising a sequence of promoter pst-sigA” and “DNA construct comprising pst-sigA” should be interpreted equally.

As used herein, the term “operably linked”, “operably encodes”, and “operably associated” are used herein interchangeably and refer to the functional linkage between a promoter and nucleic acid sequence located downstream that promoter, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which is then in turn translated into the protein encoded by the protein encoding sequence, e.g. protein having amino acid sequence set forth in SEQ ID NO: 1, 2, 3, 4 or 5.

The terms “protein encoding sequence” and “PES” are used herein interchangeably and refer to a DNA sequence encoding a protein having a particular amino acid sequence. In other words “protein encoding sequence” is translated to a peptide or a protein having the desired amino acid sequence.

According to some embodiments, the DNA construct comprises a nucleic acid sequence of at least one promoter, said sequence is selected from SEQ ID NO: 17, 14, 15, 16, and 18 (pst-sigA, skf-sigA, tua-sigA, tua-phoP, and pst-phoP promoters, respectively as disclosed in Table 1).

TABLE 1
Reference table of promoters
vs SEQ ID NOs.

| Sequence No | Promoter |
|---|---|
| SEQ ID NO: 14 | skf-sigA |
| SEQ ID NO: 15 | tua-sigA |
| SEQ ID NO: 16 | tua-phoP |
| SEQ ID NO: 17 | pst-sigA |
| SEQ ID NO: 18 | pst-phoP |

According to one embodiment, the DNA construct comprises a nucleic acid sequence SEQ ID NO: 17. According to another embodiment, the DNA construct comprises a nucleic acid sequence SEQ ID NO: 14. According to a further embodiment, the DNA construct comprises a nucleic acid sequence SEQ ID NO: 15. According to yet another embodiment, the DNA construct comprises a nucleic acid sequence SEQ ID NO: 16. According to a certain embodiment, the DNA construct comprises a nucleic acid sequence SEQ ID NO: 18. According to some embodiments, the DNA construct comprises two or more different promoters having the nucleic acid sequences select from SEQ ID NO: 17, 14, 15, 16, and 18. According to any one of the above embodiments, the promoters are disruption or tearing responsive promoters, i.e. being activated upon disruption and/or tearing of a cell.

According to other embodiments, the DNA construct comprises a sequence of a homolog of at least one of said promoters, i.e. a homolog of a nucleic acid sequence selected from SEQ ID NO: 17, 14 15, 16 and 18.

The term “homolog” as used herein refers to a DNA sequence having at least 90% identity to the parent sequence. As such, a homolog of a promoter, e.g. homolog of SEQ ID NO: 17, 14, 15, 16, and 18 has at least 90% sequence identity to sequences SEQ ID NO: 17, 14, 15, 16, and 18, respectively. According to some embodiments, the homolog has at least 95% at least 98%, or at least 99% identity to the parent sequence. According to another embodiment, the homolog has 90% to 99%, 91% to 98%, 92% to 97%, 93% to 96% or 94% to 95% identity to the parent sequence. According to the present invention, the terms “pst-sigA promoter”, “skf-sigA promoter”, “tua-sigA promoter”, tua-phoP promoter”, and “pst-phoP promoter” refers also to a homolog of the relevant promoter. According to any one of the above embodiments, the promoter homolog has the same properties and the same function as the parent promoter, i.e. being a tearing or disruption responsive promoter. Thus, according to one embodiment, the homolog is a homolog of a promoter having at least 95% sequence identity to SEQ ID NO: 17. According to another embodiment, the homolog is a homolog of a promoter having at least 95% sequence identity to SEQ ID NO:14. According to another embodiment, the homolog is a homolog of a promoter having at least 95% sequence identity to SEQ ID NO:15. According to another embodiment, the homolog is a homolog of a promoter having at least 95% sequence identity to SEQ ID NO:16. According to another embodiment, the homolog is a homolog of a promoter having at least 95% sequence identity to SEQ ID NO:18.

According to some embodiments, the DNA construct comprises 1, 2, 3, 4 or 5 of said different promoters or homologs thereof. According to one embodiment, the DNA construct comprises a promoter having the sequence of SEQ ID NO: 14. According to another embodiment, the DNA construct comprises a promoter having SEQ ID NO: 15. According to a further embodiment, the DNA construct comprises a promoter having SEQ ID NO: 16. According to a some embodiments, the DNA construct comprises a promoter having SEQ ID NO: 17. According to certain embodiments, the DNA construct comprises a promoter having SEQ ID NO: 18. According to some embodiments, the DNA construct comprises sequences of 2 promoters, e.g. SEQ ID NO: 14 and any one of SEQ ID NO: 15, SEQ ID NO: 16, SEQ ID NO: 17 or SEQ ID NO: 18; or SEQ ID NO: 15 and any one of SEQ ID NO: 16, SEQ ID NO: 17 or SEQ ID NO: 18; or SEQ ID NO: 16 and SEQ ID NO: 17 or SEQ ID NO: 18; or SEQ ID NO: 17 and SEQ ID NO: 18. In some alternative embodiments, the DNA construct comprises sequences of 3 promoters, e.g. SEQ ID NO: 14, SEQ ID NO: 15 and any one of, SEQ ID NO: 16, SEQ ID NO: 17 or SEQ ID NO: 18; SEQ ID NO: 14, SEQ ID NO: 16 and SEQ ID NO: 17 or SEQ ID NO: 18; or SEQ ID NO: 14, SEQ ID NO: 17 and SEQ ID NO: 18; SEQ ID NO: 15, SEQ ID NO: 16 and SEQ ID NO: 17 or SEQ ID NO: 18; or SEQ ID NO: 16, SEQ ID NO: 17 and SEQ ID NO: 18. In some alternative embodiments, the DNA construct comprises sequences of 4 promoters, e.g. SEQ ID NO: 14, SEQ ID NO: 15, SEQ ID NO: 16, and SEQ ID NO: 17 or SEQ ID NO: 18; SEQ ID NO: 14, SEQ ID NO: 17, SEQ ID NO: 18 and SEQ ID NO: 15 or, SEQ ID NO: 16; or SEQ ID NO: 15, SEQ ID NO: 16, SEQ ID NO: 17 and SEQ ID NO: 18. According to some embodiments, the construct comprises the sequences of all 5 promoters. According to any one of the above embodiments, the promoters may be in any order in the DNA construct relative to the 5′ terminus, e.g. when the DNA comprises the sequences of 2 promoters, such as SEQ ID NO: 17 and SEQ ID NO: 14 these promoters may be located as SEQ ID NO: 17 followed by SEQ ID NO: 14 or as SEQ ID NO: 14 followed by SEQ ID NO: 17, relative to 5′-terminus of the DNA construct. According to some embodiments, when two or more promoters are present, the may be separated by a DNA spacer, i.e. non-coding and inert DNA fragment. According to some embodiments, the DNA spacer comprises or consists of 3 to 51, 6 to 48, 9 to 42, 12 to 36, 18 to 30 or 21 to 27 nucleotides. Each one of such variants represent a separate embodiment of the invention. According to any one of the above embodiments, the term promoter refers also to a homolog of said promoter having at least 95%, at least 98% or at least 99% identity to said promoter

According to some embodiments, when 2, 3, 4, or 5 promoters or homologs thereof are present, said promoters form a cassette of promoters. Promoters is such a cassette are placed sequentially with or without a spacer.

According to any one of the above embodiments, the DNA construct comprises the promoter or combination thereof as described operably linked to a protein encoding sequence encoding a protein having an amino acid sequence selected from the group consisting of SEQ ID NOs: 1, 2, 3, 4, 5, 6, 7, 8, 9, 10, 11, 12, 13 and an analog thereof. Thus, the promoter or combination thereof is followed by a DNA sequence encoding at least one protein having an amino acid sequence selected from SEQ ID NOs: 1, 2, 3, 4, 5, 6, 7, 8, 9, 10, 11, 12 and 13 or analogs thereof.

The term “protein” as used herein includes single-chain polypeptide molecules as well as multiple-polypeptide complexes where individual constituent polypeptides are linked by covalent or non-covalent means. According to some embodiments, the term protein refers to an analog of the peptide. The term “protein analog” refers to the protein having a sequence of the parent protein in which one or more amino acids are substituted or deleted, or having one or more amino acid addition. According to some embodiment, the term “protein analog” refers to a protein having the sequence of parent protein with one or more conservative substitution as well known in the art. The term “conservative substitution” as used herein denotes the replacement of an amino acid residue by another, without altering the overall conformation and biological activity of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, according to one table known in the art, the following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W). It is well known in the art that conservative substitution does not affect the properties of the function of the protein. According to some embodiments, the protein analog as at least 90% identity to the parent amino acid sequence. As such, an analog of SEQ ID NO: 1, 2, 3, 4 and 5 has at least 90% sequence identity to respective protein. According to some embodiments, the analog has at least 95% at least 98, at least 99% identity to the parent sequence. According to another embodiment, the analog has 90% to 99%, 91% to 98%, 92% to 97%, 93% to 96% or 94 to 95% identity to the parent sequence, i.e. to SEQ ID NO: 1, 2, 3, 4 or 5. According to one embodiment, the DNA construct comprises an analog of SEQ ID NO: 1. According to another embodiment, the DNA construct comprises an analog of SEQ ID NO: 2. According to yet another embodiment, the DNA construct comprises an analog of SEQ ID NO: 3. According to certain embodiments, the DNA construct comprises an analog of SEQ ID NO: 4. According to one embodiment, the DNA construct comprises an analog of SEQ ID NO: 5. According to such embodiments, the analog has at 95%, 96%, 97%, 98% or 99% sequence identity to the parent sequence. The term “protein having the sequence set forth in SEQ ID NO. X” refers also to an analog of the respective protein, wherein X is 1, 2, 3, 4 or 5.

According to any one of the aspects and embodiments of the invention, the terms “protein comprising the amino acid sequence set forth in SEQ ID NO: X”, “protein comprising SEQ ID NO: X” and “protein having SEQ ID NO: X” are used herein interchangeably. The terms “protein consisting of the amino acid sequence set forth in SEQ ID NO: X”, “protein consisting of SEQ ID NO: X” and “protein of SEQ ID NO: X” are used herein interchangeably. The same rule holds for nucleic acid sequence. Thus the terms “nucleic acid comprising the nucleic acid sequence set forth in SEQ ID NO: X”, “nucleic acid comprising SEQ ID NO: X” and “nucleic acid having SEQ ID NO: X” are used herein interchangeably. The terms “nucleic acid consisting of the nucleic acid sequence set forth in SEQ ID NO: X”, “nucleic acid consisting of SEQ ID NO: X” and “nucleic acid of SEQ ID NO: X” are used herein interchangeably.

According to some embodiment, the term “protein analog” refers to a protein linked to a tag used for purification and/or detection of the protein as well known in the art. Non-limiting examples of such tags are poly(His) tag, chitin binding protein (CBP), maltose binding protein (MBP), Strep-tag and glutathione-S-transferase (GST). Thus in some embodiments, the protein analog refers to a protein having SEQ ID NO: 1, 2, 3, 4, and 5 linked to poly-His tag.

According to any one of the above embodiments, the proteins having the sequence set forth in SEQ ID NOs: 1-13 and analogs thereof are capable of self-assembling to form at least one fiber. The terms “fiber” and “protein fiber” are used herein interchangeably and refer to a continuous filament of discrete length made up of protein held together by intermolecular forces such as disulfide bonds, hydrogen bonds, electrostatic bonds, hydrophobic interactions, peptide strand entanglement, and covalent cross-links between side chains of proteins. According to some embodiments, the proteins having the sequence set forth in SEQ ID NOs: 1, 2, 3, 4, and 5, and analogs thereof are capable of self-assembling to form at least one fiber. According to another embodiment, these proteins are capable of assembling with each other and thereby for at least one protein fiber. Thus, the present invention provides a DNA construct, wherein the protein or proteins encoded by the PESs of the present invention are capable of self-assembling or assembling with each other to form at least one protein fiber. According to one embodiment, the protein fiber is a silk fiber. According to some embodiments, the protein analogs have the same properties and the same function as the parent protein, i.e. forms a protein fiber, and in particular silk fiber.

According to some embodiments, the term “protein having an amino acid sequence” refers to a protein comprising said amino acid sequence. According to other embodiments, this term refers also to a protein consisting of said amino acid sequence. According to a further embodiment, this term refers to a protein consisting essentially of said amino acid sequence. Thus according to some embodiments, the PESs encode for proteins comprising amino acid sequences set forth in SEQ ID NOs 1, 2, 3, 4, 5, 6, 7, 8, 9, 10, 11, 12 and 13. According to other embodiments, the PESs encode for proteins consisting of amino acid sequences set forth in SEQ ID NOs: 1, 2, 3, 4, 5, 6, 7, 8, 9, 10, 11, 12 and 13. According to yet another embodiment, the PESs encode for proteins being analogs, and particular conservative substitution analogs of protein having amino acid sequences set forth in SEQ ID NOs: 1, 2, 3, 4, 5, 6, 7, 8, 9, 10, 11, 12 and 13.

According some embodiments, the DNA construct comprises 1, 2, 3, 4, 5, 6, 7, 8, 9, 10, 11, 12 or 13 different PESs. According to some embodiments, when 2 or more, e.g., 3, 4, or 5 said different PESs are present, said PESs form a cassette of PESs. The PESs in such a cassette are placed sequentially with or without a spacer. According to some embodiments, the DNA spacer comprises or consists of 3 to 51, 6 to 48, 9 to 42, 12 to 36, 18 to 30 or 21 to 27 nucleotides. According to any one of the above embodiments, when the DNA construct comprises two or more PESs, the PESs are located in one open reading frame, i.e. in frame, and are translated to intact proteins. According to some embodiments, each PES comprise a separate open reading frame. According to another embodiment, each PES is operably linked to a copy of the promoter of the present invention. According to one embodiment, the DNA construct comprises 2 different PESs, e.g. PES encoding for a protein having amino acid sequence set forth in SEQ ID NO:1 and PES encoding for at least one of the sequences SEQ ID NO:2, 3, 4 or 5. According to another embodiment, the DNA construct comprises 3 PESs, e.g. PESs encoding for SEQ ID NO: 1 and 2 and PESs encoding for at least one of SEQ ID NO: 3, 4 or 5; or PESs encoding for SEQ ID NO: 1 and 3 and PES encoding for SEQ ID NO: 4 or 5, or PESs encoding for SEQ ID NO: 1, 4 and 5. According to further embodiments, the DNA construct comprises 4 or 5 such PESs. According to any one of the above embodiments, the PESs may be in any order within the DNA construct relative to the 5′-terminus of the DNA construct. According to some embodiments, the DNA construct comprises at least one PES encoding for a protein having an amino acid sequence set forth in SEQ ID NO:1-5 and at least one PES encoding for a protein having an amino acid sequence set forth in SEQ ID NO:6-13. Each one of such variants represent a separate embodiment of the invention.

According to certain embodiments, the DNA construct comprises one promoter and 1, 2, 3, 4, 5, 6, 7, 8, 9, 10, 11, 12 or 13 said PESs. According to some embodiments, the DNA construct comprises one promoter and 1, 2, 3, 4, or 5 said PESs. According to another embodiment, the DNA construct comprises 2 promoters and 1 to 13 or 1, 2, 3, 4, or 5 said PESs. In a further embodiment, the DNA construct comprises 3 promoters and 1 to 13 or 1, 2, 3, 4, or 5 said PESs. In certain embodiments, the DNA construct comprises 4 promoters and 1 to 13 or 1, 2, 3, 4, or 5 said PESs. In other embodiments, the DNA construct comprises 5 promoters and 1 to 13 or 1, 2, 3, 4, or 5 said PESs.

The proteins and the promoters of the present inventions may be arranged in any way well known in the art such that the protein is expressed upon activation of the promoters of the present invention. In one embodiments, when the DNA construct comprises a plurality of PESs they may form a structure of operon with one or more promoters of the present invention triggering the translation of the proteins of that operon. According to one embodiment, the promoters have the nucleic acid sequence selected from SEQ ID NO: 17, 14, 15, 16 and 18. According to other embodiments, each one of the plurality of the PES is operably linked to at least one of the promoters selected from SEQ ID NO: 17, 14, 15, 16 and 18. According to some embodiment, each one of the PES in such an arrangement is operably linked to the same or different such promoters.

According to one embodiment, the DNA construct comprises a plurality of copies of at least one promoter having the nucleic acid sequence selected from SEQ ID NO: 17, 14, 15, 16, 18 and homologue thereof. According to other embodiments, the DNA construct comprises a plurality of copies of 2, 3, 4 or 5 different promoters having the nucleic acid sequence selected from SEQ ID NO: 17, 14, 15, 16, 18 and homologues thereof. According to one embodiments, the DNA construct comprises from 1 to 100 copies of a promoter having the nucleic acid sequence selected from SEQ ID NO: 17, 14, 15, 16, 18 and homologue thereof, e.g. 10 to 90, 20 to 80, 30 to 70, 40 to 60 copies of said promoters. According to one embodiment, the DNA construct comprises 1, 2, 3, 4, 5, 6, 7, 8, 9, or 10 copies of a promoter having the nucleic acid sequence selected from SEQ ID NO: 17, 14, 15, 16, 18 and homologue thereof. According to one embodiment, the DNA construct comprises a plurality of copies of 2, 3, 4 or 5 different such promoters.

According to some embodiments, the DNA construct comprises a plurality of copies of PESs encoding the proteins of the present invention. According to one embodiment, the DNA construct comprises a plurality of copies of a PES encoding a protein having the amino acid sequences set forth in SEQ ID NOs: 1, 2, 3, 4, 5, 6, 7, 8, 9, 10, 11, 12 and 13 and analogs thereof. According to some embodiments, the DNA construct comprises a plurality of copies of each one of 2, 3, 4 or 5 different PESs. According to some embodiments, the DNA construct comprises a plurality of one or more different promoters and a plurality of one or more different PESs, such that the PESs are operably linked to the promoters and are expressed upon trigger activating said promoter.

According to some embodiments, the DNA construct comprises two or more copies of one PES. According to other embodiments, the DNA construct comprises two or more copies of two or more different PESs.

According to another embodiment, the DNA construct of the present invention comprises a promoter having the nucleic acid sequence SEQ ID NO: 17 or a homolog thereof operably linked to a protein encoding sequence encoding SEQ ID NO: 1 or an analog thereof. According to a further embodiment, the DNA construct of the present invention comprises a promoter having the nucleic acid sequence SEQ ID NO: 17 operably linked to a protein encoding sequence encoding SEQ ID NO: 1. According to certain embodiments, the DNA construct of the present invention comprises a promoter consisting of the nuclei acid sequence SEQ ID NO: 17 or a homolog thereof operably linked to a protein encoding sequence encoding SEQ ID NO: 1. Alternatively, according to such embodiment, the DNA construct comprises a protein encoding sequence encoding to an analog of SEQ ID NO: 1.

According to other such above embodiments, the DNA construct further comprises at least one promoter having a nucleic acid sequence selected from SEQ ID NO: 14, 15, 16, 18 or homolog thereof. According to one embodiment said at least one promoter is adjacent to the promoter having nucleic acid sequence SEQ ID NO: 17 or homolog thereof. According to another such alternative embodiment, the DNA construct further comprises at least one PES encoding a protein having the amino acid sequence selected from SEQ ID NOs: 2, 3, 4, 5 and analog thereof, wherein said PES. According to some embodiment, said PES is adjacent to PES encoding SEQ ID NO:1 or analog thereof, and is operably linked to said promoter(s). According to another embodiment, the DNA construct comprises nucleic acid sequence SEQ ID NO: 17, the PES encoding for SEQ ID NO: 1, at least one promoter selected from SEQ ID NO: 14, SEQ ID NO: 15, SEQ ID NO: 16, and SEQ ID NO: 18, and at least one PES encoding for amino acid sequence selected from SEQ ID NOs: 2, 3, 4 and 5. According to any one of the above embodiments, the DNA construct further comprises one or more PESs encoding a protein having the amino acid sequence selected from 6, 7, 8, 9, 10, 11, 12, and 13 being adjacent to said PES encoding SEQ ID NO:1 or analog thereof, and being operably linked to said promoter(s). According to any one of these embodiments, when the DNA construct comprises more than one promoter, they can be present in any order relative to each other. Similarly, according to another embodiment, when 2 or more different PESs are present, they can be present in any order relative to each other. According to some embodiments, a cassette of promoters is operably linked to a cassette of PESs.

According to another aspect, the present invention provides a vector comprising the DNA construct of the present invention. Thus according to some embodiments, the present invention provides a vector comprising the DNA construct comprising at least one promoter having a nucleic acid sequence selected from the group consisting of SEQ ID NO: 17, SEQ ID NO: 14, SEQ ID NO: 15, SEQ ID NO: 16, SEQ ID NO: 18 and homolog thereof operably linked to at least one protein encoding sequence, encoding a protein having the amino acid sequence selected from the group consisting of SEQ ID NOs: 1, 2, 3, 4, 5, 6, 7, 8, 9, 10, 11, 12, 13 and analog thereof. According to some embodiments, the PES encodes a protein having the amino acid sequence selected from the group consisting of SEQ ID NOs: 1, 2, 3, 4, 5, and analog thereof.

The terms “vector” and “expression vector” are used herein interchangeably and refer to any non-viral vector such as plasmid, cosmid, artificial chromosome (bacterial or yeast), or a viral vector such as virus, retrovirus, bacteriophage, or phage, binary vector in double or single stranded linear or circular form, or nucleic acid, sequence which is able to transform host cells and optionally capable of replicating in a host cell. The vector may contain an optional marker suitable for use in the identification of transformed cells, e.g., tetracycline resistance or ampicillin resistance. According to one embodiment, the vector is a plasmid. According to another embodiment, the vector is a phage or bacteriophage.

The term “plasmid” refers to circular, optionally double-stranded DNA capable of inserting a foreign DNA fragment to a cell and optionally capable of autonomous replication in a given cell. Plasmids usually contain further sequences in addition to the ones, which should be expressed, like marker genes for their specific selection and in some cases sequences for their episomal replication in a target cell. In certain embodiments, the plasmid is designed for amplification and expression in bacteria. Plasmids can be engineered by standard molecular biology techniques.

According to a further aspect, the present invention provides a cell compositing the DNA construct according to the present invention. According to some embodiments, present invention provides a cell compositing the vector of the present invention comprising said DNA construct. According to one embodiment, the vector is a plasmid. Thus, is some embodiments, the present invention provides a cell comprising the DNA construct comprising at least one promoter having a nucleic acid sequence selected from the group consisting of SEQ ID NO: 17, SEQ ID NO: 14, SEQ ID NO: 15, SEQ ID NO: 16, SEQ ID NO: 18 and homologs thereof operably linked to at least one protein encoding sequence encoding a protein having the amino acid sequence selected from the group consisting of SEQ ID NOs: 1, 2, 3, 4, 5, 6, 7, 8, 9, 10, 11, 12, 13 and analogs thereof. According to some embodiments, the PES encodes a protein having the amino acid sequence selected from the group consisting of SEQ ID NOs: 1, 2, 3, 4, 5, and analogs thereof. According to some embodiments, the cell comprises the DNA construct of the present invention comprising the sequence of SEQ ID NO: 17 promoter or homolog thereof operably linked to a protein encoding sequence encoding SEQ ID NO: 1 or analog thereof. According to one embodiment, the cell comprises the DNA construct of the present invention comprising the nucleic acid sequence SEQ ID NO: 17 operably linked to a protein encoding sequence encoding SEQ ID NO: 1. According to other such embodiments, the DNA construct further comprises at least one promoter selected from SEQ ID NO: 14, 15, 16, 18 or homolog thereof operably linked to said promoter having SEQ ID NO: 17 or homolog thereof. According to another such alternative embodiment, the DNA construct further comprises at least one PES encoding a protein having the amino acid sequence selected from SEQ ID NOs: 2, 3, 4, 5 and analog thereof, wherein said PES is adjacent to said PES encoding SEQ ID NO:1 or analog thereof, and/or being operably linked to said promoter(s).

Thus, according to some embodiments, the cells express and produce protein(s) encoded by PESs upon destruction or tear, e.g. capable of expression at least one protein comprising the amino acid sequence selected from the group consisting of SEQ ID NOs: 1, 2, 3, 4, 5, 6, 7, 8, 9, 10, 11, 12, 13 and analog thereof upon disruption or tear. According to some embodiments, the cells are capable of expressing a protein having the amino acid sequence SEQ ID NO: 1. According to another embodiment, the cells are capable of expressing a protein having the amino acid sequence SEQ ID NO: 2. According to yet another embodiment, the cells are capable of expressing a protein having the amino acid sequence SEQ ID NO: 3. According to certain embodiment, the cells are capable of expressing a protein having the amino acid sequence SEQ ID NO: 4. According to one embodiment, the cells are capable of expressing a protein having the amino acid sequence SEQ ID NO: 5. According to some embodiments, the cells are capable of expressing two or more different proteins having SEQ ID NO: 1, 2, 3, 4 and 5.

According to some embodiments, the cell is a prokaryotic cell such as bacterial cell. According to certain embodiments, the bacteria is bacteria capable of forming a biofilm with a plurality of said bacterial cells.

The cells of the present invention can be produced by any known methods such as transformation of the cells with the vector such as plasmid, or infecting the cells with a viral vector.

The terms “biofilm” and “bacterial biofilm” are used herein interchangeably and refers to a community of bacteria cells being contained within an extracellular matrix produced by the bacteria.

According to some embodiments, the bacteria is gram-positive bacteria. Non-limiting examples of gram-positive bacteria according to the present invention are e.g. Bacillus spp, Listeria monocytogenes, Staphylococcus spp, and lactic acid bacteria, including Lactobacillus plantarum and Lactococcus lactis. According to some embodiments, the bacteria belongs to Bacillus species. According to more particular embodiment, the bacteria is Bacillus subtilis, Bacillus pumilus, or Bacillus licheniformis.

According to another embodiment, the bacteria are gram-negative species such as Escherichia coli, or Pseudomonas spp. and in particular Pseudomonas putida, Pseudomonas fluorescens or Pseudomonas aeruginosa.

According to any one of the above embodiments, the bacteria is modified bacteria lacking genes causing to pathogenesis.

According to any one of the above embodiments, the biofilm of the present invention comprising the cells of the present invention is capable of producing protein fibers, more specifically silk fibers upon disruption or tear of the biofilm. Therefore, the biofilm of the present invention is capable of closing disruption or tear in the biofilm.

According to another aspect, the present invention provides a biofilm comprising a plurality of cells according to the present invention. According to one embodiment, the present invention provides a biofilm comprising a plurality of cells comprising a DNA construct or a vector comprising at least one promoter having a nucleic acid sequence selected from the group consisting of SEQ ID NO: 17, SEQ ID NO: 14, SEQ ID NO: 15, SEQ ID NO: 16, SEQ ID NO: 18 and homolog thereof, said promoter is operably linked to at least one protein encoding sequence, encoding for a protein having the amino acid sequence selected from the group consisting of SEQ ID NOs: 1, 2, 3, 4, 5, 6, 7, 8, 9, 10, 11, 12, 13 and analog thereof. According to some embodiments, the cells are bacterial cells such as of Bacillus spp, in particular Bacillus subtilis. According to some embodiments, the biofilm comprises a plurality of cells of the present invention. According to some embodiments, the biofilm is attached to a surface of a matter, incorporated within, or intertwines with a matter. According to some embodiment, the biofilm is adsorbed to the surface, intertwined with or interlaced with a fabric or textile product. According to some embodiments, cells of the biofilm are capable of expressing a protein having the amino acid sequence SEQ ID NO: 1. According to another embodiment, cells of the biofilm are capable of expressing a protein having the amino acid sequence SEQ ID NO: 2. According to yet another embodiment, cells of the biofilm are capable of expressing a protein having the amino acid sequence SEQ ID NO: 3. According to certain embodiment, cells of the biofilm are capable of expressing a protein having the amino acid sequence SEQ ID NO: 4. According to one embodiment cells of the biofilm are capable of expressing a protein having the amino acid sequence SEQ ID NO: 5. According to some embodiments, cells of the biofilm are capable of expressing two or more different proteins having SEQ ID NO: 1, 2, 3, 4 and 5. According to some embodiments, the cells of the biofilm express said proteins upon disruption or tear of said biofilm. According to such embodiment, the proteins form a protein fiber upon expression.

According to another aspect, the present invention provides a fabric incorporating the biofilm according to the present invention. The terms “incorporating” and “incorporated” are used herein interchangeably and have the meaning of adsorbed or attached to the surface as well as included within, intertwined or interlaced with the matter. As used herein the term “adsorbed” and its variations mean a physical intervention of the sorbed material into the absorbent without chemical change of the absorbed material, and in particular means bonding of the sorbed material onto the surfaces of the adsorbent. The term “interlace” herein is broadly used to describe the situation when at least one filament or fiber interweaves with the other filaments, e.g., one of the filaments passes first above the crossed filament and then passes under the next crossed filament. Thus according to one embodiment, the present invention provides a fabric or textile adsorbed with the biofilm of the present invention, i.e. the biofilm is attached to the surface of the fabric or textile. In other embodiments, the present invention provides a fabric or textile intertwined or interlaced with said biofilm, i.e. the biofilm is located within, interlaced with and intertwined with the matter, e.g. intertwined with the fibers of fabric or textile. Thus in one embodiment, the fabric comprises the cells or the biofilm comprising the cells of the present invention. According to some embodiments, the cells comprise a DNA construct or a vector comprising at least one promoter having a nucleic acid sequence selected from the group consisting of SEQ ID NO: 17, SEQ ID NO: 14, SEQ ID NO: 15, SEQ ID NO: 16, SEQ ID NO: 18 and homolog thereof, said promoter is operably linked to at least one protein encoding sequence, encoding for a protein having the amino acid sequence selected from the group consisting of SEQ ID NOs: 1, 2, 3, 4, 5, 6, 7, 8, 9, 10, 11, 12, 13 and analog thereof. According to one embodiment, the fabric comprises cells such as bacteria or a biofilm comprising a DNA construct comprising a promoter having SEQ ID NO: 17 operably linked to a protein encoding sequence encoding for a protein having the amino acid sequence SEQ ID NO: 1. Therefore, the fabric comprising the bacteria or the biofilm of the present invention forms protein fibers upon tear or disruption of said fabric, thereby self-healing said fabric.

The term “fabric” refers to a material made through weaving, knitting, spreading, crocheting, or bonding that may be used in production of further goods. According to some embodiments, fabric material is in a form of a woven material, a non-woven material or combinations thereof. The term “fabric” includes also the term “textile”. The term “textile” refers to any material made of interlacing fibers, which may be natural or artificial.

According to one embodiment, the fabric is a textile article. According to some embodiments, the textile is made of natural fibers. According to one embodiment, the textile is made of synthetic fibers. According to a further embodiment, the textile is made of a blend of natural and synthetic fibers. According to some embodiments, the fabric is selected from cotton, silk, wool, cashmere, rayon wool, linen, hemp, ramie, jute, rayon, nylon, polyester, acrylic, spandex, olefin fiber, polyester viscose, polyester wool, modacrylic and olefin. According to another embodiment, the textile is selected from cotton, silk, wool, cashmere, linen, hemp, ramie, and jute.

According to any one of the above embodiments, the biofilm of the present invention produces protein fibers, such as silk fibers upon disruption or tear of the biofilm. According to some embodiments, the fibers are interlaced with the teared fibers of the fabric or the textile, adhered to or intertwined with the biofilm. According to some embodiments, the biofilm protrudes protein fibers upon disruption or tearing of the fabric and consequently of the biofilm and therefore are capable of covering, enclosing, closing or enveloping the tear or disruption.

Thus, according to some embodiments, the present invention provides a self-fixing, self-recovering or self-healing fabric or textile comprising the biofilm of the present invention. In some embodiments, the present invention provides self-recovering clothes comprising the biofilm of the present invention. In some embodiments, the present invention provides a self-healing textile incorporating a biofilm comprising a plurality of cells, said cells comprising the DNA construct of the present invention or the vector of the present invention.

According to one aspect, the present invention provides a method of preparing self-recovering fabric, textile or cloth, said method comprises contacting said fabric, textile or clothes with the bacteria of the present invention.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

Having now generally described the invention, the same will be more readily understood through reference to the following examples, which are provided by way of illustration and are not intended to be limiting of the present invention.

### EXAMPLES

### Materials and methods

### Bacterial Strains and Growth Conditions

B. subtilis (strain NCIB3610) harboring a chromosomally encoded GFP reporter gene and Chloramphenicol (CM) resistance as well as the Wild Type (WT) strains were a kind gift from Ilana Kolodkin-Gal (Weizmann Institute of Science). For biofilm formation, bacteria were cultured in Minimal medium (MSgg) as previously described at 23° C. for 72 hours.

### Fabrics

Several types of fabrics (silk, 100% cotton, synthetics, mixes, Gütermann threads and others) were purchased from Gudes and The Sewing Center LTD. (Israel). Fabrics were cleaned by washing in filtered deionized water and sterilized by soaking in 70% ethanol followed by in-plate UV irradiation. Fabrics were stored at room temperature and humidity in closed sterilized culture plates.

### Microscopy and Image Analysis

Structural analysis of fabric and biofilm architecture and bacterial growth and integration into the fabric were measured using PhenomWorld ProX SEM with EDS module. Samples were prepared with minimum manipulations. Fresh and unfixed samples were dried in a low vacuum desiccator for ˜20 minutes, until biofilms were dry but not cracked.

Images were analyzed using ImageJ (Fiji) software. For analysis all images used were formatted to 8-bit binary (grayscale). SEM information bars were cropped out of the figures before analysis. Histograms and surface plots were done using default parameters and all actions were applied on all figures identically.

### Transcriptome Response to Tear

Biofilms were subjected to mechanical tear by a custom-built moving array of sterilized, round-tip stainless steel needles (200 micron tip diameter, 1 mm tip-to-tip distance) introduced into the biofilm to a depth of 4 mm to ensure simultaneous uniform tearing in as many points as possible. RNA was extracted from biofilms 5 minutes following tearing using FastRNA PRO™ BLUE kit (MP Biomedicals). Experiments were done in triplicates and quality and quantity of RNA was evaluated using spectrophotometry on a Nanodrop 2000 instrument and bioanalyzer (Agilent 2100). Library preparation (TruSeq RNA without the oligo-dT stage) and sequencing (SR 60 v4 High Output) were performed at the Nancy and Stephen Grand Israel National Center for Personalized Medicine, Weizmann Institute of Science, Israel.

### Bioinformatic Analysis

Quality control on RNA sequence reads was done using FastQC. Adapters were removed using Cutadapt, discarding reads with less than 40 bases after adapter trimming. Reads with more than 50% polyA/T were removed using a custom-written script. Counting was done using HTSeq and gene annotation was based on Ensembles B. subtilis GTF. Differential expression analysis was done using DESeq2 (1.6.3), with no independent filtering and beta prior. Raw p-values were adjusted for multiple testing using FDR(BH).

### Plasmid and Strain Construction

pBS3Clux-[RFP] integration plasmid for gram positive bacteria was a kind gift from Daniel R. Zeigler (The Bacillus Genetic Stock Center). RFP gene was replaced with pst-sigA (SEQ ID NO: 17) promoter. Genome integration transformations of B. subtilis into sacA locus were carried out using the competent strain DK1042, an identical strain to NCIB3610 except for a single point mutation that inactivates comI, a naturally-occurring plasmid-borne competence-suppressing gene, and increases competence 100-fold. Competent cultures were grown in diluted modified competence (MC) medium as previously described and plated on LB plates containing 5 ug/ml chloramphenicol to select for transformants. Integration of plasmids into sacA locus of the B. subtilis genome was checked with colony PCR.

### Luciferase Assay

Luciferase activity of strain harboring chromosomally encoded PSEQ ID NO: 17-luxABCDE was assayed using a SynergyHTX multi-mode reader from BioTek® (Winooski, Vt., USA). The reader was controlled using the software Gen5. Culture volumes were 100 μl per well in a bioluminescence-compatible 96-well plate, and incubation occurred at 23° C. for 72 hours. In all wells biofilms were formed. Positive control strain harboring a constitutive strong promoter Pveg was used to adjust sensitivity for optimum results. Plate was monitored for luminescence prior to mechanical tear. Once biofilms were subjected to tear (as described above), luminesce was monitored for 30 minutes in 4-minute intervals. Experiment was done in triplicates.

### Gene Design and Synthesis

Glycine/Serine/Alanine-rich segments (termed spsegI to spsegV) from silk genes sequenced from Australian raspy crickets were selected for their chemical properties and assembly potential. For initial expression, protein segments were synthesized de novo using E. coli codon optimization and cloned into a Clontech pBE-S vector (a system optimized for Bacillus secreted proteins). For expression in arthropod cells (S2 drosophila cells), genes were recoded for eukaryotic expression and recloned into the pMT/BiP/V5-HisA vector, containing the N-terminal signal sequence from the insect BiP gene, a C-terminal V5 epitope, and a C-terminal 6His tag for purification.

### Flow Cytometry

S2 cell count and viability assays were done on a BD Accuri™ C6 flow cytometer. Cells were checked routinely every 3 days, before subculturing and transfections.

For analysis, S2 cultures were diluted 1:10 into PBS and viewed in forward scatter/side scatter channels. For viability analysis, propidium iodide (PI) was added to a final concentration of 0.1 ng/μL and cells were vortexed briefly. Silk gene transfection success and protein production were evaluated by intracellular flow cytometry as follow: cells were fixed with 2% formaldehyde, then perforated by a brief incubation in frozen 100% methanol. Cells were washed with FX buffer (0.1% w/v bovine serum albumin, 0.05% w/v sodium azide in PBS, pH 7.4), and incubated with primary (anti-V5 epitope) and secondary antibodies with washes in between.

### Protein Expression and Purification

Silk segments were first expressed in E. coli to assess assembly into fibers, and extracted using a commercial kit. Fibers were analyzed visually and by SEM. S2 cells were used for large scale purification, and silk segments were purified on a Ni-NTA column in an AKTA-Start instrument and evaluated by SDS-PAGE and western blot. Western blotting was performed on a Bio-Rad blotting system using commercially available reagents and standard protocols; membranes were developed using Novex HRP Chromo kit. Purified protein was dialyzed overnight into PBS on 12,500 Da molecular weight cutoff dialysis tubes, concentrated with 3 sequential runs on Amicon 0.5 mL 10,000 Da cutoff tubes, and acidified with 17 M acetic acid to a pH of ˜5.8.

### Example 1. Compatibility of a Bacterial Biofilm with Fabric

To study the feasibility of hybridizing the fabric with the a biofilm we cultured Bacillus subtilis biofilms embedded inside pieces of fabric of different origins (animal source such as wool and silk; plant source such as cotton and flax; mineral source such as asbestos; and synthetic such as nylon, polyester, and acrylic) and weaving patterns (fiber diameter and fiber density). All fabrics were compatible with biofilm growth and maintenance, with minor detected differences in viability or activity between groups. Interestingly, there was a clear correlation between fabric architecture and biofilm appearance; hybrids with less dense fabrics exhibited rough-surfaced, disordered biofilms, and ones within denser fabrics exhibiting the opposite phenotype. This dependency suggests that the fabric serves as a structural framework or scaffold for the biofilm, and highlights the possibility of designing specific fabrics to achieve desired biofilm phenotypes and growth patterns.

### Example 2. Elucidation of Gene Expressed Upon Exposure of Biofilm to Mechanical Tear

In order to configure the response/synthesis role of the biofilm, the response of the biofilm to mechanical tear was mapped. While other responses have been previously reported, the specific response to mechanical strain and tear, a likely natural scenario in bacterial evolution, has not. For this, total RNA was extracted from B. subtilis biofilms 5 min after subjecting them to mechanical tear, sequenced and analyzed to obtain the transcriptome response and identify tear-responsive elements. Rather than a single tear across a biofilm, and in order to maximize the signal, ˜1,000 of small lateral tears were induced in the entire biofilm (average of ˜3 tears per mm² of biofilm) using a custom-built array of metal needles positioned at high density and movable on the XYZ axes.

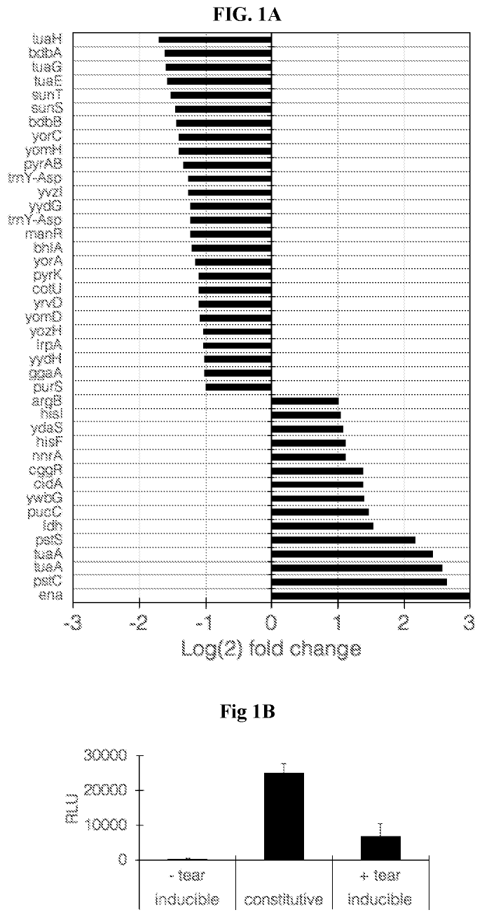

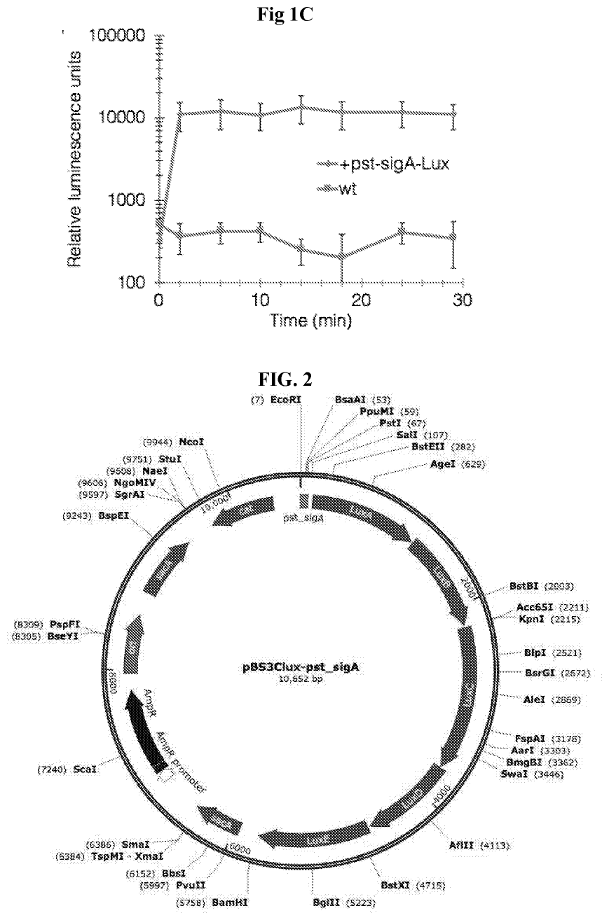

Transcriptome analysis highlighted specific pathways involved in the response to tearing (FIG. 1A), particularly cell wall remodeling (teichuronic acid and peptidoglycan biosynthesis) and cell division (phosphate uptake, nucleotide and aminoacyl-tRNA biosynthesis). Tearing also induced activation of the sigma M regulon, which has been shown to operate in response to cell wall stress induced by antibiotics and other chemical stimuli. Interestingly, population control genes such as skf (sporulation killing factor) and sdpA/B (sporulation delaying proteins) were inhibited, suggesting a potential disinhibition for purposes of population regrowth. These patterns were highly reproducible in independent experiments. Based on these findings, 5 promoters (pst-sigA, skf-sigA, tua-sigA, tua-phoP, and pst-phoP) were identified and selected as candidate tearing-induced drivers of fabric synthesis (the sequences are presented in Table 2).

TABLE 2
DNA sequences of pst-sigA, skf-sigA, tua-sigA, tua-phoP,
and pst-phoP promoters.

| SEQ ID NO | Promoter | Sequence |
|---|---|---|
| SEQ ID NO: 14 | skf-sigA | AATTTTTAGGATAATATACAAAATCCCCCTTACTT |
| | | CGACAATTGCAATCTGGTATTATCGTATCGCAT |
| SEQ ID NO: 15 | tua-sigA | ATACCATTTACATCCAATTAACATCCGTCTGCTAA |
| | | ACTGACTGGCATAGG |
| SEQ ID NO: 16 | tua-phoP | ATTCACACTTCTTAACATACCATTTACATCCAATTA |
| | | ACATCCGTC |
| SEQ ID NO: 17 | pst-sigA | TTCGGTTCAAACCCTTTTTACATAGAACCTTTACTC |
| | | TATACGTGTAGGAC |
| SEQ ID NO: 18 | pst-phoP | CACTGATTTACAAAACCTTAACATTCGGTTCAAAC |
| | | CCTTTTTACATAGAAC |

All 5 promoters showed at least 8-fold increase in expression upon stimulus while maintaining minimal expression unstimulated. Test drivers were constructed in which each of the 5 promoters was placed to control expression of the luxABCDE operon (the map of a representative plasmid is shown in FIG. 2), and biofilms transformed with these drivers responded well to tearing. A representative response to tearing as measured by luminescence measurement is presented in FIG. 1B and C.

### Example 3. Expression of Silk Fiber Upon Tear of a Biofilm

Next, we turned to designing the synthetic part of the system. The choice of genes for fabric synthesis was guided by mechanistic simplicity: a single gene, and the ability to self-assemble into a functional fiber under specific conditions. Arthropod silks have been known for millennia and are still considered industrial benchmarks today. However, silks from spider species or from the silkworm Bombyx mori require complex weaving organs, making them unsuitable for the purposes of the present design. For this reason, silk from other sources was examined. Raspy crickets (Gryllacrididae) produce silk for building leaf shelters. Recently, several genes encoding cricket silk were cloned from the cricket labial glands, and their partial sequences include alanine/glycine/serine-rich repeats typical of silk proteins from other species (Walker, Andrew A., et al. “Silk from crickets: a new twist on spinning.” PloS one 7.2 (2012): e30408).

In order to evaluate the suitability of these proteins for the synthesis of a silk rod, segments of these protein sequences (termed spsegI/II/III/IV/N and corresponding to SEQ ID NOs 2, 1, 3, 4 and 5, respectively) were selected and each was fused to histidine tags for expression vector construction.

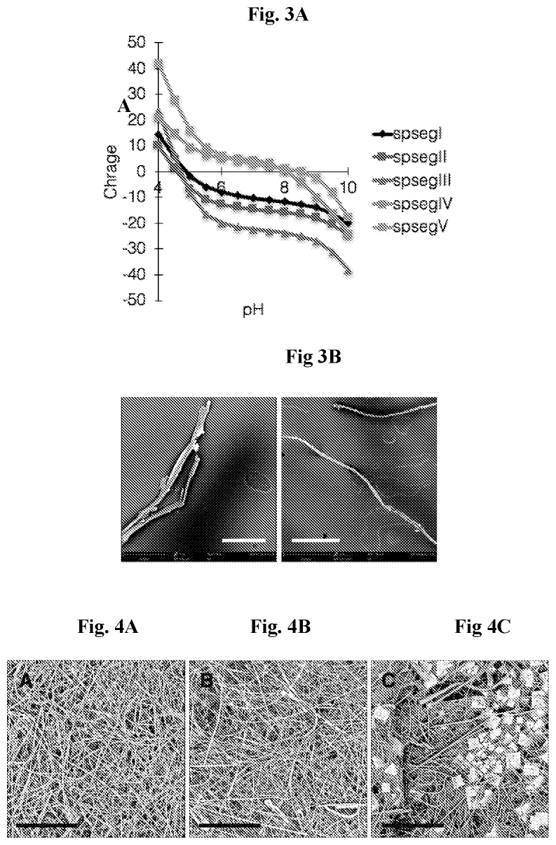

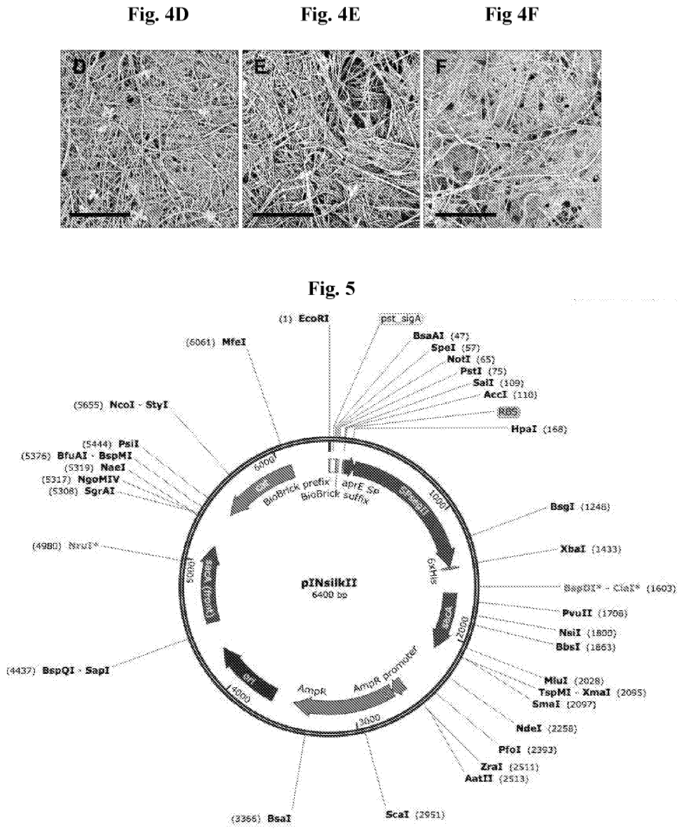

Isoelectric points of the protein segments were calculated, with an interesting distribution into two groups, an ‘acidic’ group containing 3 proteins with pI at ˜5.0, and an ‘alkaline’ group containing the remaining 2 proteins with pI at ˜8.0 (FIG. 3A). The proteins were expressed in both insect and bacterial cell; in both cases showing efficient assembly upon cumulative acidification and dehydration (increasing protein concentration) (FIG. 3B and FIG. 4) into fibers of a mean diameter of 10 um, with an elastic modulus of 4.54 GPa and tensile strength of 617 MPa.

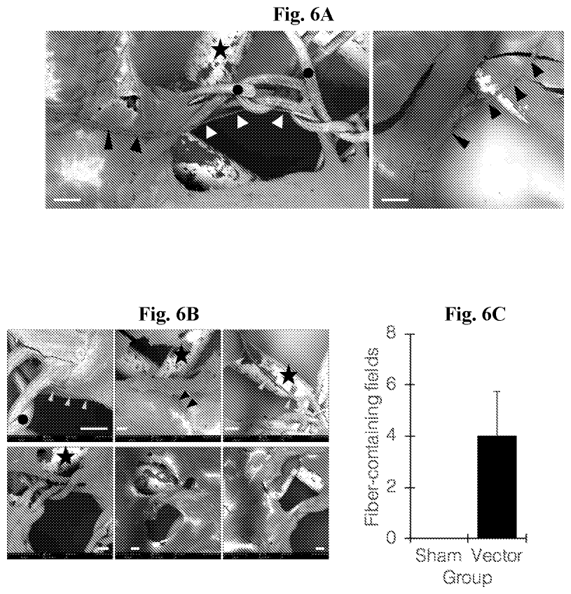

Finally, a selected protein segment was placed under the control of a selected promoter (see e.g. FIG. 5), and the fabric-biofilm hybrids were prepared using B. subtilis transformed with the constructed vector. Five (5) min following tearing the hybrid on the liquid-air interface and under slightly acidic conditions (pH ˜6.0), we observed assembled single fibers, originating within ±20 um from the rim of the tear region (FIG. 6A). Interestingly, silk protein assembly occurred mostly along and around fabric fibers, suggesting that the assembly process is more efficient on the fiber surface, hence the fabric serves as scaffold or guide for the process. Hybrids made with wildtype biofilms were torn as well, without any apparent response (FIG. 6B, C).

Although the present invention has been described herein above by way of preferred embodiments thereof, it can be modified, without departing from the spirit and nature of the subject invention as defined in the appended claims.

1-23. (canceled)

24. A DNA construct comprising a nucleic acid sequence of at least one promoter operably linked to at least one protein encoding sequence, wherein said at least one promoter has a nucleic acid sequence selected from the group consisting of SEQ ID NO: 17, 14, 15, 16, 18 and homologs thereof, and said at least one protein encoding sequence encodes a protein having an amino acid sequence selected from the group consisting of SEQ ID NOs: 1, 2, 3, 4, 5, 6, 7, 8, 9, 10, 11, 12, 13 and analogs thereof, wherein the homolog and the analog have at least 90% sequence identity to the parent sequence.

25. The DNA construct of claim 24, comprising a promoter having the nucleic acid sequence SEQ ID NO: 17 or a homolog thereof operably linked to a protein encoding sequence encoding a protein having the sequence set forth in SEQ ID NO: 1 or an analog thereof.

26. The DNA construct of claim 25, comprising a promoter having the nucleic acid sequence SEQ ID NO: 17 or a homolog thereof and at least one additional promoter having the nucleic acid sequence selected from SEQ ID NO: 14, 15, 16, 18 and homologs thereof.

27. The DNA construct of claim 24, wherein the DNA construct comprises two or more different protein encoding sequences.

28. The DNA construct of claim 24, wherein said encoded protein or proteins are capable of self-assembling or assembling with each other to form at least one protein fiber.

29. A vector comprising the DNA construct of claim 24.

30. The vector of claim 29, wherein the vector is a plasmid or phage.

31. A cell comprising the DNA construct or the vector of claim 29.

32. The cell of claim 31, wherein the cell is a bacterial cell.

33. The cell of claim 32, capable of forming a biofilm with a plurality of said cells.

34. The cell of claim 31, wherein the cell is capable of expressing at least one protein having an amino acid sequence selected from the group consisting of SEQ ID NOs: 1, 2, 3, 4, 5, 6, 7, 8, 9, 10, 11, 12, 13 and analog thereof, wherein said analog have at least 90% sequence identity to the parent sequence.

35. A biofilm comprising a plurality of cells of claim 31.

36. The biofilm of claim 35, wherein said biofilm expresses at least one of said encoded proteins upon disruption or tearing of said biofilm.

37. The biofilm of claim 35, wherein said biofilm forms one or more protein fiber upon disruption of said biofilm.

38. A fabric, wherein said fabric incorporates a plurality of cells of claim 31.

39. A fabric, wherein said fabric incorporates the biofilm of claim 35, said biofilm is adsorbed to the surface of, or interlaced with the fabric.

40. The fabric of claim 38, wherein the fabric is a textile made of natural fibers, synthetic fibers or a blend thereof.

41. The fabric of claim 40, wherein the textile is selected from the group consisting of cotton, silk, wool, cashmere, linen, hemp, ramie, and jute.

42. The fabric of claim 40, wherein said biofilm forms at least one protein fiber upon tearing or disruption of the fabric.

43. A self-healing textile comprising a biofilm of claim 35.
